(19) United States
(12) Patent Application Publication
(10) Pub. No.: US 2025/0204864 A1
(43) Pub. Date: Jun. 26, 2025

(54) PHYSIOLOGICAL STATE ESTIMATION SYSTEM, PHYSIOLOGICAL STATE ESTIMATION METHOD, AND NON-TRANSITORY COMPUTER READABLE MEDIUM

(71) Applicant: TOYOTA JIDOSHA KABUSHIKI KAISHA, Toyota-shi Aichi-ken (JP)

(72) Inventors: Keiji HAYASHI, Toyota-shi Aichi-ken (JP); Hitoshi YAMADA, Toyota-shi Aichi-ken (JP); Yuhei YAMAGUCHI, Toyota-shi Aichi-ken (JP)

(73) Assignee: TOYOTA JIDOSHA KABUSHIKI KAISHA, Toyota-shi Aichi-ken (JP)

(21) Appl. No.: 18/989,864

(22) Filed: Dec. 20, 2024

(30) Foreign Application Priority Data
Dec. 25, 2023 (JP) 2023-218505

(51) Int. Cl. A61B 5/00

(52) U.S. Cl. CPC A61B 5/7235; A61B 5/7275

(57) ABSTRACT

A physiological state estimation system according to this embodiment includes: a waveform information acquisition unit configured to acquire brain waves and pulse waves; a filtering unit configured to filter the acquired brain waves and pulse waves in at least one predetermined frequency band; a transformation unit configured to perform Hilbert transformation on the brain waves and pulse waves filtered in the frequency band; and an instantaneous value calculation unit configured to calculate instantaneous values including an instantaneous logarithmic amplitude corresponding to a logarithm of an amplitude term of a complex waveform equation obtained by performing Hilbert transformation, and an instantaneous frequency corresponding to a time differential value of a phase term of a complex waveform equation.

### CROSS REFERENCE TO RELATED APPLICATIONS

This application is based upon and claims the benefit of priority from Japanese patent application No. 2023-218505, filed on Dec. 25, 2023, the disclosure of which is incorporated herein in its entirety by reference.

### BACKGROUND

The present disclosure relates to a physiological state estimation system, a physiological state estimation method, and a physiological state estimation program.

Patent Literature 1 discloses a biological signal analysis method for calculating feature amounts from biological signals such as brain waves, pulse waves, and myoelectricity using a Lyapunov exponent by assuming chaotic nature to obtain quantitative information on a living body. The biological signal analysis method disclosed in Patent Literature 1 calculates feature amounts at a high speed by using a high-frequency region.
[Patent Literature 1] Japanese Unexamined Patent Application Publication No. 2002-017687
[Non-Patent Literature 1]W Zong, T Heldt, G B Moody and R G Mark, “An Open-source Algorithm to Detect Onset of Arterial Blood Pressure Pulses”, Computers in Cardiology 2003; 30:259-262. [online], [searched on Dec. 14, 2021], Internet <https://lcp.mit.edu/pdf/Zong03a.pdf>

### SUMMARY

It is required to provide a method for estimating a physiological state from a biological signal with a high accuracy.

The present disclosure has been made in order to solve the aforementioned problem, and provides a physiological state estimation system, a physiological state estimation method, and a physiological state estimation program for estimating a physiological state with a high accuracy.

A physiological state estimation system according to this embodiment includes: a waveform information acquisition unit configured to acquire brain waves and pulse waves; a filtering unit configured to filter the acquired brain waves and pulse waves in at least one predetermined frequency band; a transformation unit configured to perform Hilbert transformation on the brain waves and pulse waves filtered in the frequency band; an instantaneous value calculation unit configured to calculate instantaneous values including an instantaneous logarithmic amplitude corresponding to a logarithm of an amplitude term of a complex waveform equation obtained by performing Hilbert transformation and an instantaneous frequency corresponding to a time differential value of a phase term of the complex waveform equation; a distribution calculation unit configured to calculate a probability density distribution of the instantaneous values and calculate feature amounts including a mean value and a variance value of the probability density distribution; an extraction unit configured to extract the instantaneous values when |μₑ−μ_{b}|<(σₑ+σ_{b})/1 is met, where a mean value of the instantaneous frequency of the brain waves in the predetermined frequency band is denoted by μₑ, a variance value of the instantaneous frequency of the brain waves in the predetermined frequency band is denoted by σₑ, a mean value of the instantaneous frequency of the pulse waves in the predetermined frequency band is denoted by μ_{b}, a variance value of the instantaneous frequency of the pulse waves in the predetermined frequency band is denoted by σ_{b}, and a parameter of an integer equal to or greater than 3 is denoted by η; and a physiological state estimation unit configured to estimate a physiological state from the feature amounts of the instantaneous values that have been extracted. According to this configuration, it is possible to estimate the physiological state with a high accuracy.

In the aforementioned physiological state estimation system, η of the parameter may be 3. According to this configuration, it is possible to estimate the physiological state with a higher accuracy.

A physiological state estimation method according to this embodiment includes: a waveform information acquisition step of acquiring brain waves and pulse waves; a filtering step of filtering the acquired brain waves and pulse waves in at least one predetermined frequency band; a transformation step of performing Hilbert transformation on the brain waves and pulse waves filtered in the frequency band; an instantaneous value calculation step of calculating instantaneous values including an instantaneous logarithmic amplitude corresponding to a logarithm of an amplitude term of a complex waveform equation obtained by performing Hilbert transformation and an instantaneous frequency corresponding to a time differential value of a phase term of the complex waveform equation; a distribution calculation step of calculating a probability density distribution of the instantaneous values and calculating feature amounts including a mean value and a variance value of the probability density distribution; an extraction step of extracting the instantaneous values when |μₑ−μ_{b}|<(σₑ+σ_{b})/η is met, where a mean value of the instantaneous frequency of the brain waves in the predetermined frequency band is denoted by μₑ, a variance value of the instantaneous frequency of the brain waves in the predetermined frequency band is denoted by σₑ, a mean value of the instantaneous frequency of the pulse waves in the predetermined frequency band is denoted by μ_{b}, a variance value of the instantaneous frequency of the pulse waves in the predetermined frequency band is denoted by σ_{b}, and a parameter of an integer equal to or greater than 3 is denoted by η; and a physiological state estimation step of estimating a physiological state from the feature amounts of the instantaneous values that have been extracted. According to this configuration, it is possible to estimate the physiological state with a high accuracy.

A physiological state estimation program according to this embodiment causes a computer to execute: a waveform information acquisition step of acquiring brain waves and pulse waves; a filtering step of filtering the acquired brain waves and pulse waves in at least one predetermined frequency band; a transformation step of performing Hilbert transformation on the brain waves and pulse waves filtered in the frequency band; an instantaneous value calculation step of calculating instantaneous values including an instantaneous logarithmic amplitude corresponding to a logarithm of an amplitude term of a complex waveform equation obtained by performing Hilbert transformation and an instantaneous frequency corresponding to a time differential value of a phase term of the complex waveform equation; a distribution calculation step of calculating a probability density distribution of the instantaneous values and calculating feature amounts including a mean value and a variance value of the probability density distribution; an extraction step of extracting the instantaneous values when |μₑ−μ_{b}|<(σₑ+σ_{b})/η is met, where a mean value of the instantaneous frequency of the brain waves in the predetermined frequency band is denoted by μₑ, a variance value of the instantaneous frequency of the brain waves in the predetermined frequency band is denoted by σₑ, a mean value of the instantaneous frequency of the pulse waves in the predetermined frequency band is denoted by μ_{b}, a variance value of the instantaneous frequency of the pulse waves in the predetermined frequency band is denoted by σ_{b}, and a parameter of an integer equal to or greater than 3 is denoted by η; and a physiological state estimation step of estimating a physiological state from the feature amounts of the instantaneous values that have been extracted. According to this configuration, it is possible to estimate the physiological state with a high accuracy.

According to this embodiment, it is possible to provide a physiological state estimation system, a physiological state estimation method, and a physiological state estimation program estimating a physiological state with a high accuracy.

The above and other objects, features and advantages of the present disclosure will become more fully understood from the detailed description given hereinbelow and the accompanying drawings.

### BRIEF DESCRIPTION OF DRAWINGS

FIG. 1 is a diagram illustrating a method for measuring biological waves according to a first embodiment;

FIG. 2 is a graph illustrating biological waves according to the first embodiment, in which the horizontal axis indicates a time and the vertical axis indicates a pulse interval and a pulse amplitude;

FIG. 3 is a graph illustrating biological waves according to the first embodiment, in which the horizontal axis indicates a time and the vertical axis indicates an intensity;

FIG. 4 is a graph illustrating biological waves according to the first embodiment, in which the horizontal axis indicates a time and the vertical axis indicates an intensity;

FIG. 5 is a graph illustrating biological waves according to the first embodiment, in which the horizontal axis indicates a time and the vertical axis indicates an intensity;

FIG. 6 is a graph illustrating a waveform obtained by performing Fourier transformation on brain waves and pulse waves according to the first embodiment, in which the horizontal axis indicates a frequency and the vertical axis indicates an intensity;

FIG. 7 is a graph illustrating a waveform obtained by performing Fourier transformation on brain waves and pulse waves according to the first embodiment, in which the horizontal axis indicates a frequency and the vertical axis indicates an intensity;

FIG. 8 is a diagram illustrating a waveform obtained by performing filtering processing on biological waves in a plurality of frequency bands according to the first embodiment, in which the horizontal axis indicates a time and the vertical axis indicates an intensity;

FIG. 9 is a diagram illustrating a waveform in which biological waves are filtered in a plurality of frequency bands according to the first embodiment, in which the horizontal axis indicates a time and the vertical axis indicates an intensity;

FIG. 10 is a diagram illustrating a waveform in which biological waves are filtered in the plurality of frequency bands according to the first embodiment, in which the horizontal axis indicates a time and the vertical axis indicates an intensity;

FIG. 11 is a diagram showing a complex waveform equation obtained by performing Hilbert transformation on biological waves on a complex plane according to the first embodiment;

FIG. 12 is a graph illustrating a probability density distribution of an instantaneous frequency of brain waves in an a frequency band with eyes open according to the first embodiment, in which the horizontal axis indicates a frequency and the vertical axis indicates a density;

FIG. 13 is a graph illustrating a probability density distribution of an instantaneous frequency of pulse waves in the α frequency band with eyes open according to the first embodiment, in which the horizontal axis indicates a frequency and the vertical axis indicates a density;

FIG. 14 is a graph illustrating a probability density distribution of an instantaneous phase difference of brain waves and pulse waves in the α frequency band with eyes open according to the first embodiment, in which the horizontal axis indicates a phase difference and the vertical axis indicates a density;

FIG. 15 is a graph illustrating a probability density distribution of an instantaneous frequency of brain waves in the α frequency band with eyes open according to the first embodiment, in which the horizontal axis indicates a frequency and the vertical axis indicates a density;

FIG. 16 is a graph illustrating a probability density distribution of an instantaneous frequency of pulse waves in the α frequency band with eyes open according to the first embodiment, in which the horizontal axis indicates a frequency and the vertical axis indicates a density;

FIG. 17 is a graph illustrating a probability density distribution of an instantaneous phase difference of brain waves and pulse waves in the α frequency band with eyes open according to the first embodiment, in which the horizontal axis indicates a phase difference and the vertical axis indicates a density;

FIG. 18 is a graph illustrating a probability density distribution of an instantaneous frequency of brain waves in the α frequency band with eyes closed according to the first embodiment, in which the horizontal axis indicates a frequency and the vertical axis indicates a density;

FIG. 19 is a graph illustrating a probability density distribution of an instantaneous frequency of pulse waves in the α frequency band with eyes closed according to the first embodiment, in which the horizontal axis indicates a frequency and the vertical axis indicates a density;

FIG. 20 is a graph illustrating a probability density distribution of an instantaneous phase difference of brain waves and pulse waves in the α frequency band with eyes closed according to the first embodiment, in which the horizontal axis indicates a phase difference and the vertical axis indicates a density;

FIG. 21 is a graph illustrating a probability density distribution of an instantaneous frequency of brain waves in the α frequency band with eyes closed according to the first embodiment, in which the horizontal axis indicates a frequency and the vertical axis indicates a density;

FIG. 22 is a graph illustrating a probability density distribution of an instantaneous frequency of pulse waves in the α frequency band with eyes closed according to the first embodiment, in which the horizontal axis indicates a frequency and the vertical axis indicates a density;

FIG. 23 is a graph illustrating a probability density distribution of an instantaneous phase difference of brain waves and pulse waves in the α frequency band with eyes closed according to the first embodiment, in which the horizontal axis indicates a phase difference and the vertical axis indicates a density;

FIG. 24 is a graph illustrating feature amounts when the eyes are open and when the eyes are closed according to the first embodiment, in which the horizontal axis indicates a time and the vertical axis indicates a feature amount of an instantaneous frequency, a feature amount of an instantaneous logarithmic amplitude, and a degree of concentration;

FIG. 25 is a diagram illustrating a complex waveform equation in each frequency band in a three dimensional manner according to the first embodiment;

FIG. 26 is a diagram illustrating a complex waveform equation in each frequency band in a three dimensional manner according to the first embodiment;

FIG. 27 is a block diagram illustrating a physiological state estimation apparatus according to the first embodiment; and

FIG. 28 is a flowchart illustrating a physiological state estimation method according to the first embodiment.

### DESCRIPTION OF EMBODIMENTS

Embodiments of the present disclosure will now be described. However, the claimed disclosure is not limited to the following embodiments. Moreover, not all of the configurations described in the embodiments are required for solving the problem. For the sake of clarity, the following descriptions and drawings are omitted and simplified as appropriate. In each drawing, the same elements have the same reference signs, and repeated descriptions have been omitted as appropriate.

### First Embodiment

First, waveform information on a living body according to this embodiment will be described. The living body is, for example, a subject. A physiological state of the subject is estimated from the waveform information on the living body. The waveform information on the living body includes biological waves. The biological waves include, for example, pulse-related waves and brain-related waves. The pulse-related waves may include pulse waves and pulse interval waves. The brain-related waves may include brain waves, carotid artery waves and cerebral blood flow waves. The biological waves may include waveform information other than the pulse-related waves and the brain-related waves as long as the biological waves are waveform information on the living body. The pulse-related waves may include waveform information other than pulse waves and pulse interval waves as long as the pulse-related waves are waveform information related to a pulse. The brain-related waves may include waveform information other than brain waves, carotid artery waves, and cerebral blood flow waves as long as the brain-related waves are waveform information related to a brain. Hereinafter, brain waves, pulse waves, and pulse interval waves will be described as the biological waves.

### <Measurement of Biological Waves>

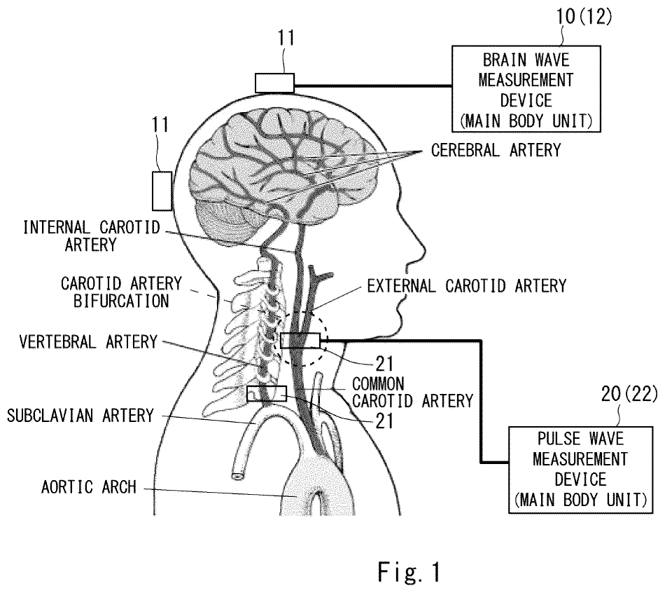

Of the biological waves, a method for measuring brain waves, pulse waves, and pulse interval waves will be described. FIG. 1 is a diagram illustrating a method for measuring biological waves according to the first embodiment. As shown in FIG. 1, a measurement device that measures biological waves may be, for example, a brain wave measurement device 10 and a pulse wave measurement device 20.

The brain wave measurement device 10 acquires, as waveform information on a living body, brain waves of a subject. The brain wave measurement device 10 includes a sensor 11 and a main body unit 12. The sensor 11 is placed in each of a plurality of parts by the 10-20 method or the like, and the main body unit 12 performs measurement simultaneously. The sensor 11 is attached, for example, onto a scalp of a subject's head, and senses information on the brain waves of the subject from the outside of the living body. The information on the brain waves of the subject is, for example, a voltage. The sensor 11 may sense, as information on the brain waves of the subject, other than the voltage, a current, a magnetic field or the like. The sensor 11 is attached to the living body in a non-invasive manner.

The sensor 11 outputs information on the sensed brain waves to the main body unit 12 of the brain wave measurement device 10. The main body unit 12 of the brain wave measurement device 10 measures a temporal change in a voltage or the like output from the sensor 11. The sensor 11 is connected to the main body unit 12 by a wired or wireless communication line.

The pulse wave measurement device 20 measures pulse waves of the subject as waveform information on the living body. The pulse waves are waveform information on the living body formed by a pulse interval, a blood output, and physical characteristics of blood vessels. The pulse wave measurement device 20 includes a sensor 21 and a main body unit 22. The pulse wave measurement device 20 may be either a photoelectric device such as a photoplethysmogram or a piezoelectric device. When the photoelectric type device is used, a near-infrared light in which the light wavelength is from 800 to 1000 nm may be used. The sensor 21 is attached, for example, onto a skin on the neck (carotid artery bifurcations) or cervical vertebrae (vertebral arteries) of the subject, and measures information on the pulse waves flowing into the brain of the subject from the outside of the living body.

Specifically, the sensor 21 may be disposed in at least one of a part near the right and left carotid artery bifurcations or a part near the right and left vertebral arteries. The information on the pulse waves is, for example, a pulse pressure. The sensor 21 may sense, besides the pulse pressure, an amount of the blood flow as the information on the pulse waves. The sensor 21 is attached to a living body in a non-invasive manner.

The sensor 21 outputs the sensed information on the pulse waves to the main body unit 22 of the pulse wave measurement device 20. The main body unit 22 of the pulse wave measurement device 20 measures a temporal change of a pulse pressure or the like output from the sensor 21. The sensor 21 is connected to the main body unit 22 by a wired or wireless communication line.

### <Time-Series Data of Biological Waves>

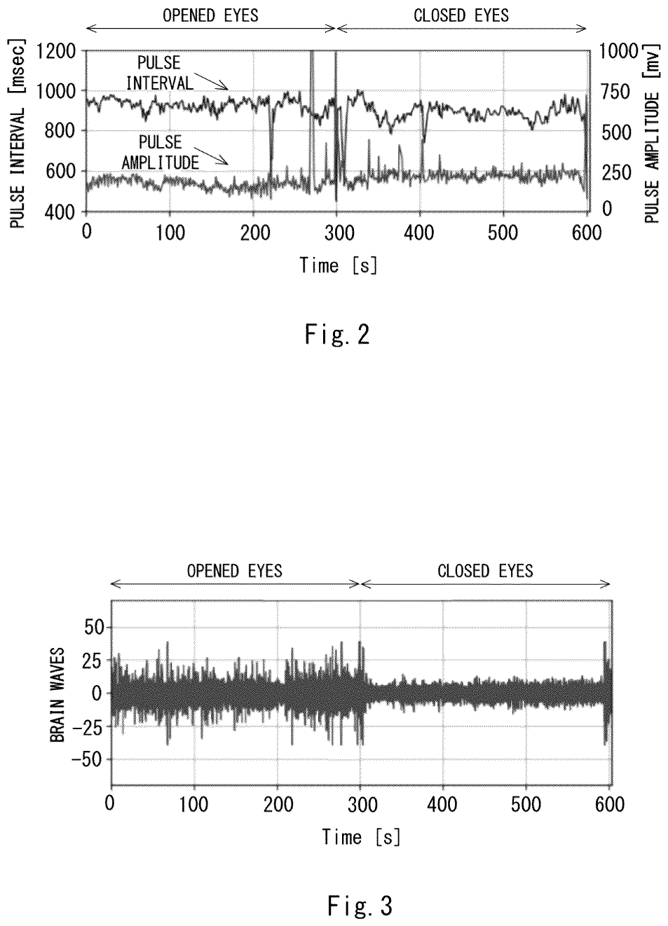

Next, time-series data of biological waves will be described. FIG. 2 is a graph illustrating biological waves according to the first embodiment, in which the horizontal axis indicates a time and the vertical axis indicates a pulse interval and a pulse amplitude. FIG. 3 is a graph illustrating biological waves according to the first embodiment, in which the horizontal axis indicates a time and the vertical axis indicates an intensity. FIG. 2 shows a pulse interval and a pulse amplitude of pulse waves acquired from right and left carotid artery bifurcations as an example of biological waves. FIG. 3 shows brain waves acquired from the frontal lobe as an example of biological waves. As shown in FIGS. 2 and 3, brain waves and pulse waves form time-series data. In FIGS. 2 and 3, 0-300 seconds show measurement results with eyes opened, which is, for example, measurement results in a case where a subject is taking a quiz with eyes closed. 300-600 seconds show measurement results with eyes closed, which is, for example, measurement results in a case where the subject is listening to music with eyes closed.

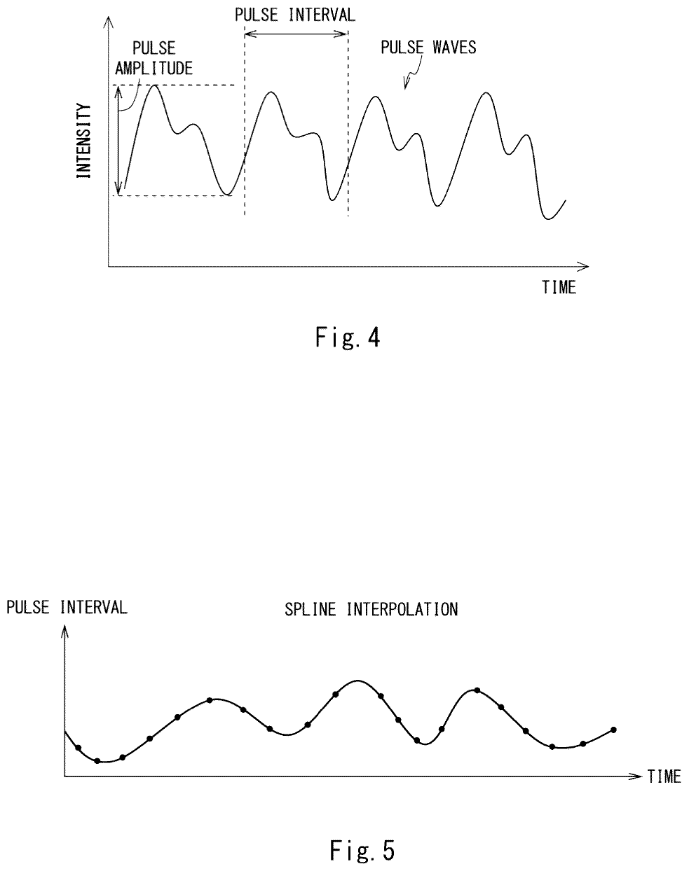

FIGS. 4 and 5 are graphs illustrating biological waves according to the first embodiment, in which the horizontal axis indicates a time and the vertical axis indicates an intensity. FIG. 4 shows pulse waves for defining pulse interval waves from pulse waves, and a pulse amplitude from pulse waves. FIG. 5 shows pulse interval waves as an example of biological waves. The pulse interval waves will be obtained according to the following procedure. First, time-series data of pulse waves shown in FIG. 4 is obtained by calculating a pulse interval (PPI: Peak-Peak Interval) using a pulse wave rising position detection algorithm presented in Non-Patent Literature 1. Next, rising times of the pulse waves and the pulse interval are plotted and subjected to spline interpolation, whereby pulse interval waves as shown in FIG. 5 can be obtained. As shown in FIG. 5, pulse interval waves also form time-series data. While biological waves other than the brain waves, the pulse waves, and the pulse interval waves also form time-series data, only brain waves, pulse waves, and pulse interval waves are shown in FIG. 5. In this embodiment, a physiological state of the living body is estimated from the time-series data of the biological waves.

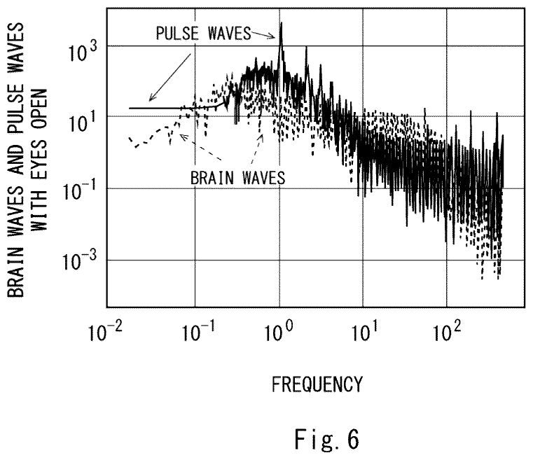

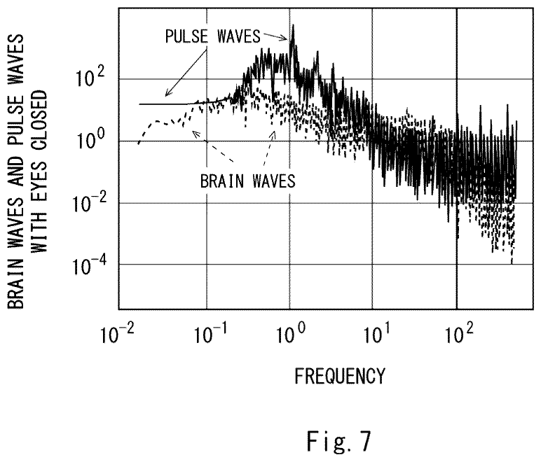

FIGS. 6 and 7 are graphs each illustrating a waveform obtained by performing Fourier transformation on brain waves and pulse waves according to the first embodiment, in which the horizontal axis indicates a frequency and the vertical axis indicates an intensity. FIG. 6 shows brain waves and pulse waves with eyes open in FIGS. 2 and 3, and FIG. 7 shows brain waves and pulse waves with eyes closed in FIGS. 2 and 3.

### <Frequency Band>

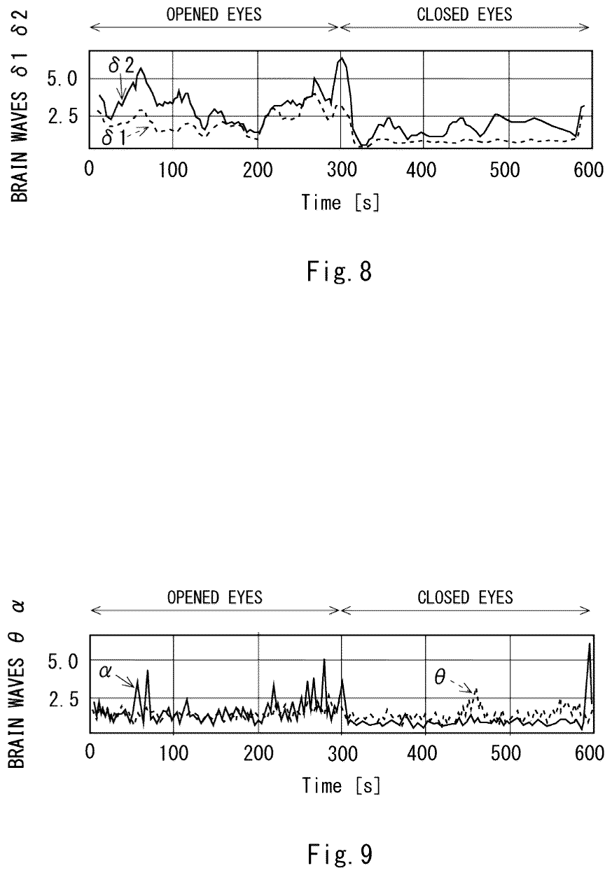

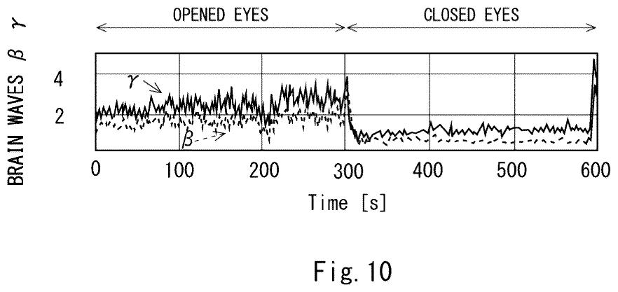

In this embodiment, biological waves are subjected to filtering processing in a plurality of frequency bands in a predetermined period. The predetermined period is, for example, a period in which it can be regarded that a physiological state is in a constant state. The frequency band is, for example, VLF2, VLF1, LF, HF, δ1, δ2, θ, α, β, γ or the like. FIGS. 8-10 are diagrams illustrating waveforms obtained by performing filtering processing on biological waves in a plurality of frequency bands according to the first embodiment, in which the horizontal axis indicates a time and the vertical axis indicates an intensity. FIG. 8 shows, for example, brain waves filtered in frequency bands of δ1 and δ2. FIG. 9 shows, for example, brain waves filtered in frequency bands of θ and α. FIG. 10 shows, for example, brain waves filtered in frequency bands of β and γ.

As shown in FIGS. 8 to 10, the biological waves filtered in each frequency band form time-series data. The brain waves may be filtered in frequency bands of, besides the above ones, VLF2, VLF1, LF, and HF. Biological waves, not only brain waves, may be subjected to filtering processing in a plurality of frequency bands. In this example, however, only brain waves are shown. In FIGS. 8-10, 0-300 seconds indicate measurement results with eyes open and 300-600 seconds indicate measurement results with eyes closed. In the following, each frequency band will be described.

VLF2 is, for example, a frequency band from 0.004 to 0.015 Hz. The central frequency of VLF2 is, for example, 0.01 Hz. VLF2 is a frequency band indicating the functions of autonomic nerves, such as thermoregulation, digestion, excretion, reproduction, immunity, etc.

VLF1 is, for example, a frequency band from 0.015 to 0.04 Hz. The central frequency of VLF1 is, for example, 0.0300 Hz. VLF1 is also a frequency band indicating the functions of autonomic nerves, such as thermoregulation, digestion, excretion, reproduction, immunity, etc.

LF is, for example, a frequency band from 0.04 to 0.15 Hz. The central frequency of LF is, for example, 0.1 Hz. LF is a frequency band of a blood pressure fluctuation band indicating a function of blood pressure.

HF is, for example, a frequency band from 0.15 to 0.40 Hz. The central frequency of HF is, for example, 0.30 Hz. HF is a frequency band of a respiratory fluctuation band indicating a function of respiration.

δ1 is, for example, a frequency band from 0.4 to 1.5 Hz. The central frequency of δ1 is, for example, 1.0 Hz.

δ2 is, for example, a frequency band from 1.5 to 4 Hz. The central frequency of δ2 is, for example, 3.0 Hz.

θ is, for example, a frequency band from 4.0 to 8.0 Hz. The central frequency of θ is, for example, 5.0 Hz.

α is, for example, a frequency band from 8.0 to 12.0 Hz. The central frequency of α is, for example, 10.0 Hz.

β is, for example, a frequency band from 12.0 to 30.0 Hz. The central frequency of β is, for example, 20.0 Hz.

γ is, for example, a frequency band larger than 30 Hz.

### <Complex Transformation>

The filtered biological waves in each frequency band are transformed, for example, into a complex waveform equation by Hilbert transformation.

Specifically, time-series data φₖ⁽ⁿ⁾(t) of biological waves in each frequency band is transformed into a complex waveform equation as shown in the following Equation (1) by Hilbert transformation.

$${φ}_{k}^{( n )} ( t ) = exp ( {a}_{k}^{( n )} ( t ) + i {ψ}_{k}^{( n )} ( t ) ) \qquad ( 1 )$$

Here, k indicates each frequency band. That is, k=1, 2, . . . indicates a frequency band such as VLF2, VLF1, LF, HF, δ1, δ2, θ, α, β, γ, . . . etc. n denotes each biological wave. That is, n=1, 2, . . . indicates one of pulse-related waves or brain-related waves. In particular, brain waves, pulse waves, and pulse interval waves are respectively expressed by φₖ^{(e)}(t), φₖ^{(b)}(t), and φₖ^{(r)}(t) using n=e, n=b, and n=r as shown in Equations (2)-(4).

$${φ}_{k}^{( e )} ( t ) = exp ( {a}_{k}^{( e )} ( t ) + i {ψ}_{k}^{( e )} ( t ) ) \qquad ( 2 )$$

$${φ}_{k}^{( b )} ( t ) = exp ( {a}_{k}^{( b )} ( t ) + i {ψ}_{k}^{( b )} ( t ) ) \qquad ( 3 )$$

$${φ}_{k}^{( r )} ( t ) = exp ( {a}_{k}^{( r )} ( t ) + i {ψ}_{k}^{( r )} ( t ) ) \qquad ( 4 )$$

From Equation (1), the following Equations (5)-(7) can be obtained.

$${a}_{k}^{( n )} ( t ) \qquad ( 5 )$$

$${ψ}_{k}^{( n )} ( t ) \qquad ( 6 )$$

$${ω}_{k}^{( n )} ( t ) = d {ψ}_{k}^{( n )} ( t ) / dt \qquad ( 7 )$$

Note that the biological waves before being filtered in each frequency band may be expressed by a linear combination of the frequency band k, as shown in Equation (8).

$${φ}^{( n )} ( t ) = \sum exp ( {a}_{k}^{( n )} ( t ) + i {ψ}_{k}^{( n )} ( t ) ) \qquad ( 8 )$$

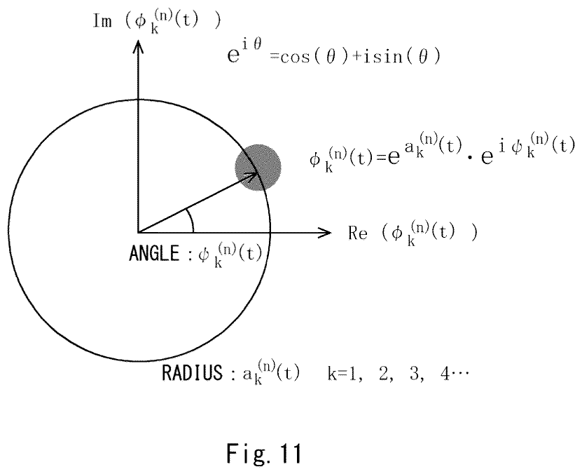

FIG. 11 is a diagram showing a complex waveform equation obtained by performing Hilbert transformation on biological waves on a complex plane according to the first embodiment. As shown in FIG. 11, biological waves φₖ⁽ⁿ⁾(t) that are filtered in the k frequency band and are shown by a complex waveform equation can be expressed on a complex plane. When the horizontal axis on the complex plane indicates Re(lnφₖ⁽ⁿ⁾(t)) and the vertical axis on the complex plane indicates Im(lnφₖ⁽ⁿ⁾(t)), aₖ⁽ⁿ⁾(t) indicates a radius centered on the origin and ψₖ⁽ⁿ⁾(t) indicates an angle with the horizontal axis. Here, k=1, 2, 3, 4 . . . indicates each frequency band.

### <Definitions of Instantaneous Values>

Next, instantaneous values will be described. As shown in Equation (5), the logarithm aₖ⁽ⁿ⁾(t) of the amplitude term of the complex waveform equation is called an instantaneous logarithmic amplitude of biological waves in a k frequency band, and ψₖ⁽ⁿ⁾(t) in Equation (6) is called an instantaneous phase of biological waves in the k frequency band. The time differential value ωₖ⁽ⁿ⁾(t) of the instantaneous phase in Equation (7) is called an instantaneous frequency of biological waves in the k frequency band. The instantaneous logarithmic amplitude corresponds to a logarithm of an amplitude term of a complex waveform equation obtained by performing Hilbert transformation. The instantaneous phase corresponds to a phase term of the complex waveform equation obtained by performing Hilbert transformation. The instantaneous frequency corresponds to a time differential value of the phase term of the complex waveform equation.

Further, as shown in Equation (9), a difference between respective instantaneous phases in two different biological waves is referred to as an instantaneous phase difference θₖ^{(x, y)}(t). Here, (x, y) indicates that instantaneous phases of two different biological waves (n=x) and (n=y) are used.

$${θ}_{k}^{( x , y )} ( t ) = {ψ}_{k}^{( x )} ( t ) - {ψ}_{k}^{( y )} ( t ) \qquad ( 9 )$$

Here, the two different biological waves are expressed by Equations (10) and (11).

$${φ}_{k}^{( x )} ( t ) = exp ( {a}_{k}^{( x )} ( t ) + i {ψ}_{k}^{( x )} ( t ) ) \qquad ( 10 )$$

$${φ}_{k}^{( y )} ( t ) = exp ( {a}_{k}^{( y )} ( t ) + i {ψ}_{k}^{( y )} ( t ) ) \qquad ( 11 )$$

In particular, as shown in Equations (12)-(14), the instantaneous phase difference between the instantaneous phase of the pulse waves and the instantaneous phase of the pulse interval waves is denoted by θₖ^{(b, r)}(t), an instantaneous phase difference between the instantaneous phase of the brain waves and the instantaneous phase of the pulse waves is denoted by θₖ^{(e, b)}(t), and the instantaneous phase difference between the instantaneous phase of the brain waves and the instantaneous phase of the pulse interval waves is denoted by θₖ^{(e-r)}(t).

$${θ}_{k}^{( b , r )} ( t ) = {ψ}_{k}^{( b )} ( t ) - {ψ}_{k}^{( r )} ( t ) \qquad ( 12 )$$

$${θ}_{k}^{( e , b )} ( t ) = {ψ}_{k}^{( e )} ( t ) - {ψ}_{k}^{( b )} ( t ) \qquad ( 13 )$$

$${θ}_{k}^{( e , r )} ( t ) = {ψ}_{k}^{( e )} ( t ) - {ψ}_{k}^{( r )} ( t ) \qquad ( 14 )$$

The instantaneous values include the instantaneous logarithmic amplitude aₖ⁽ⁿ⁾(t), the instantaneous phase ψₖ⁽ⁿ⁾(t), the instantaneous frequency θₖ⁽ⁿ⁾(t), and the instantaneous phase difference θₖ^{(x, y)}(t).

### <Distribution of Instantaneous Values>

By measuring the instantaneous values at a predetermined time interval, a probability density distribution of instantaneous values can be obtained. Accordingly, it is possible to obtain the probability density distribution of the instantaneous amplitude aₖ⁽ⁿ⁾(t), the probability density distribution of the instantaneous phase ψₖ⁽ⁿ⁾(t), the probability density distribution of the instantaneous frequency ωₖ⁽ⁿ⁾(t), and the probability density distribution of the instantaneous phase difference θₖ^{(x, y)}(t).

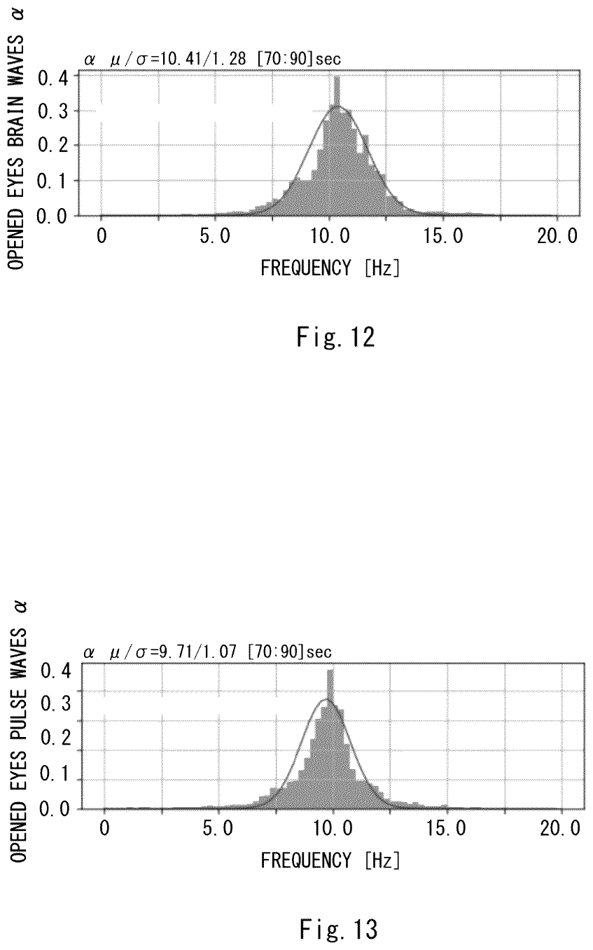

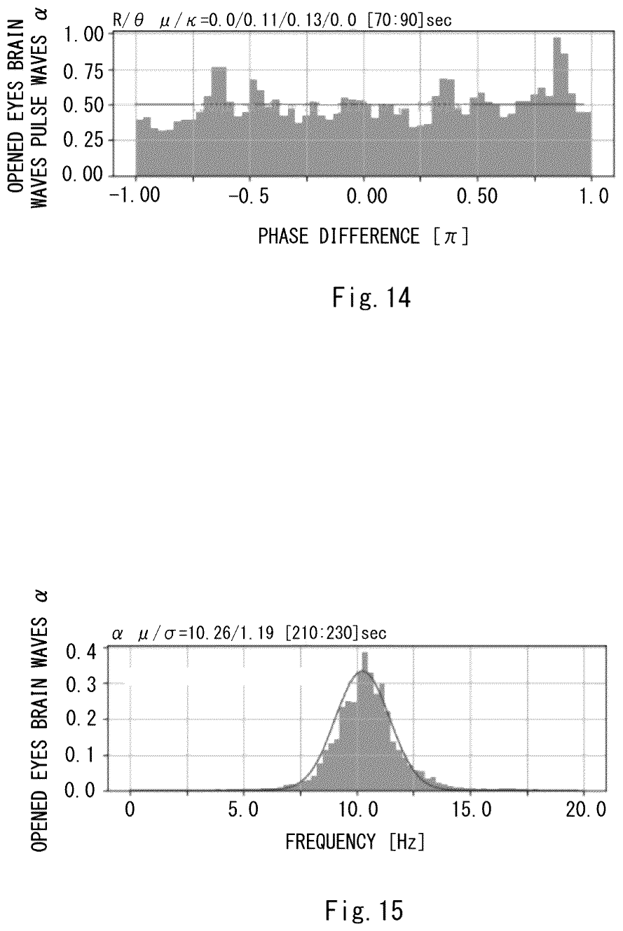

FIG. 12 is a graph illustrating a probability density distribution of an instantaneous frequency of brain waves in an a frequency band with eyes open according to the first embodiment, in which the horizontal axis indicates a frequency and the vertical axis indicates a density. FIG. 13 is a graph illustrating a probability density distribution of an instantaneous frequency of pulse waves in the α frequency band with eyes open according to the first embodiment, in which the horizontal axis indicates a frequency and the vertical axis indicates a density. FIG. 14 is a graph illustrating a probability density distribution of an instantaneous phase difference of brain waves and pulse waves in the α frequency band with eyes open according to the first embodiment, in which the horizontal axis indicates a phase difference and the vertical axis indicates a density.

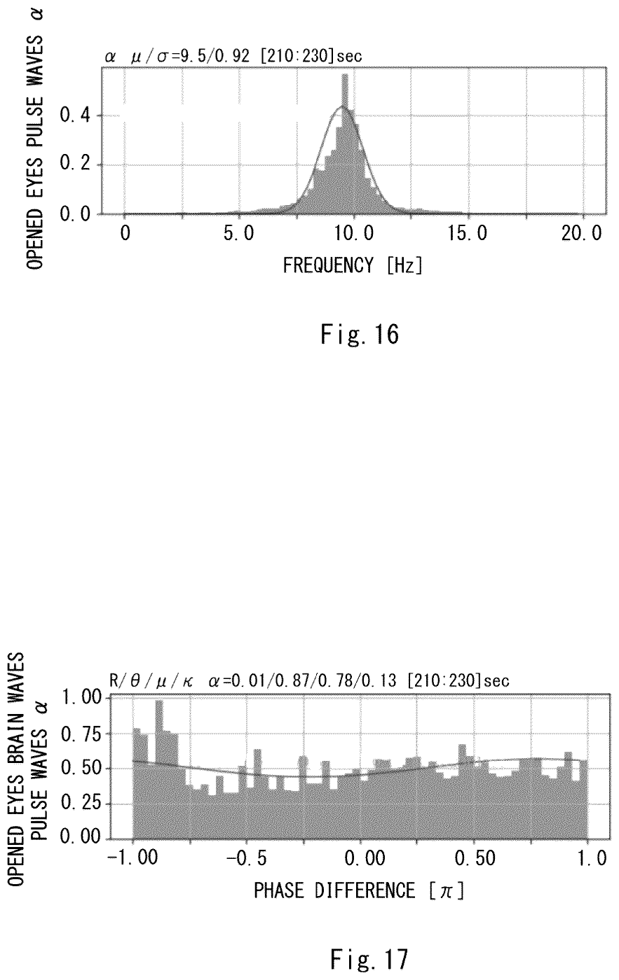

FIG. 15 is a graph illustrating a probability density distribution of an instantaneous frequency of brain waves in the α frequency band with eyes open according to the first embodiment, in which the horizontal axis indicates a frequency and the vertical axis indicates a density. FIG. 16 is a graph illustrating a probability density distribution of an instantaneous frequency of pulse waves in the α frequency band with eyes open according to the first embodiment, in which the horizontal axis indicates a frequency and the vertical axis indicates a density. FIG. 17 is a graph illustrating a probability density distribution of an instantaneous phase difference of brain waves and pulse waves in the α frequency band with eyes open according to the first embodiment, in which the horizontal axis indicates a phase difference and the vertical axis indicates a density.

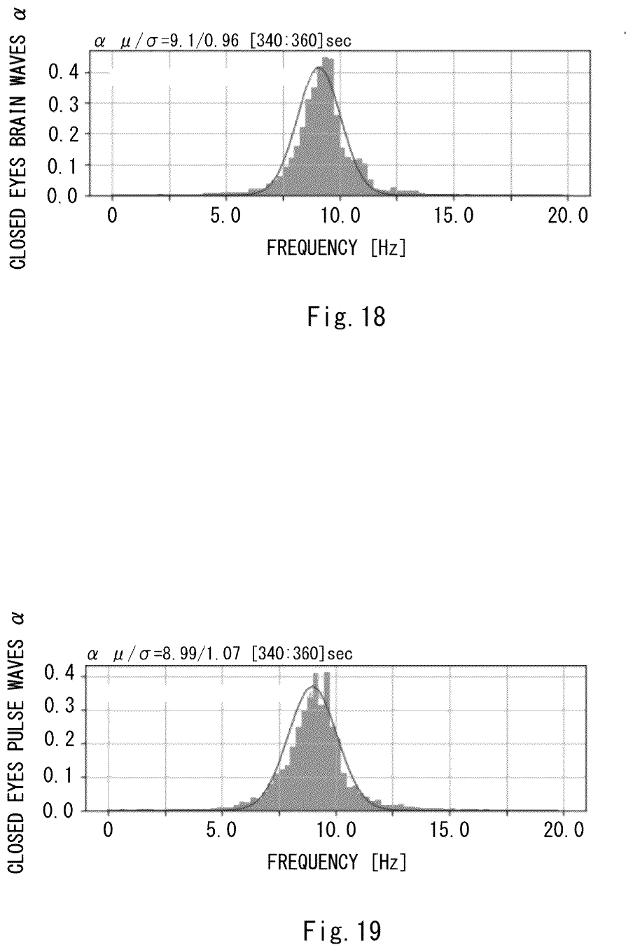

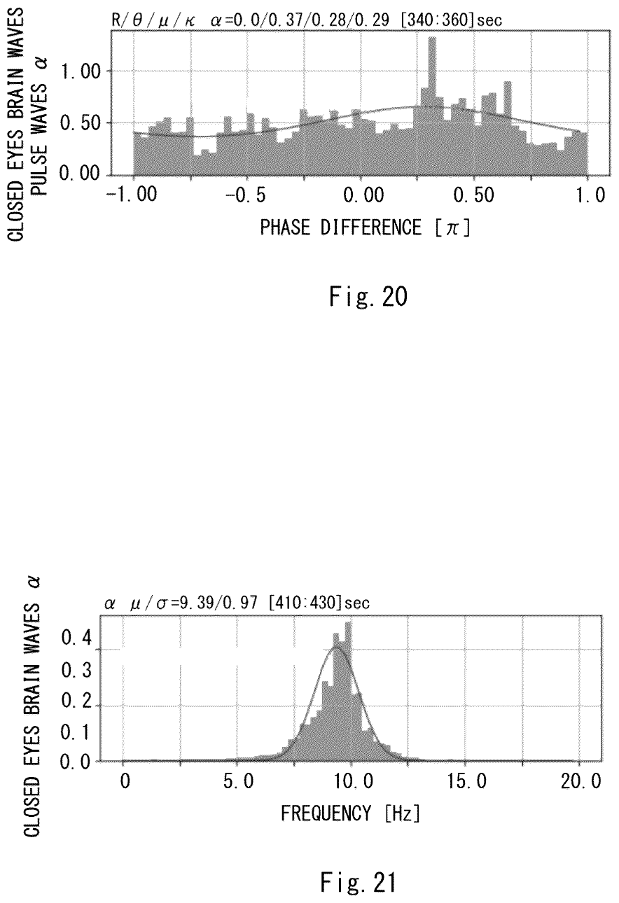

FIG. 18 is a graph illustrating a probability density distribution of an instantaneous frequency of brain waves in the α frequency band with eyes closed according to the first embodiment, in which the horizontal axis indicates a frequency and the vertical axis indicates a density. FIG. 19 is a graph illustrating a probability density distribution of an instantaneous frequency of pulse waves in the α frequency band with eyes closed according to the first embodiment, in which the horizontal axis indicates a frequency and the vertical axis indicates a density. FIG. 20 is a graph illustrating a probability density distribution of an instantaneous phase difference of brain waves and pulse waves in the α frequency band with eyes closed according to the first embodiment, in which the horizontal axis indicates a phase difference and the vertical axis indicates a density.

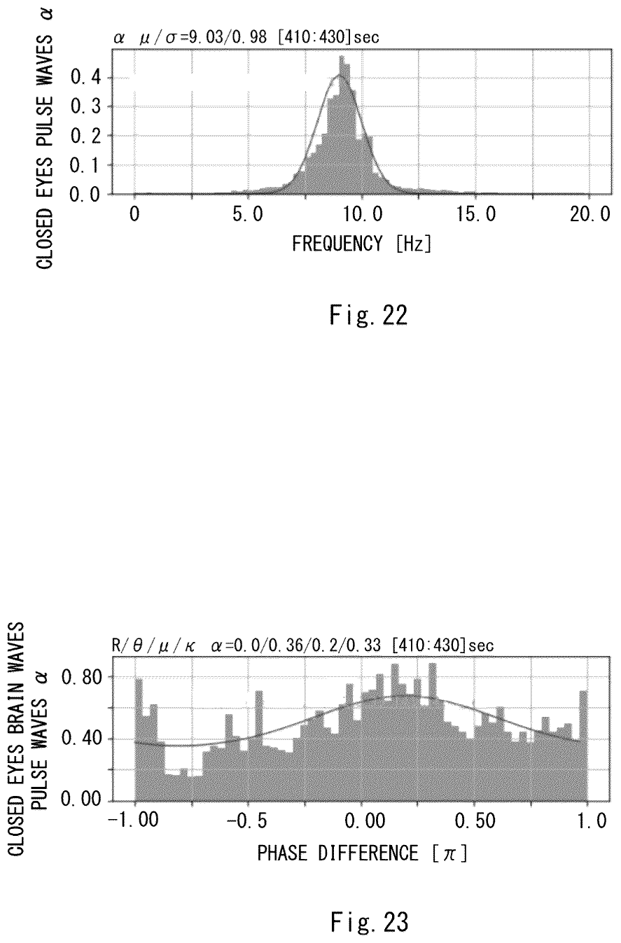

FIG. 21 is a graph illustrating a probability density distribution of an instantaneous frequency of brain waves in the α frequency band with eyes closed according to the first embodiment, in which the horizontal axis indicates a frequency and the vertical axis indicates a density. FIG. 22 is a graph illustrating a probability density distribution of an instantaneous frequency of pulse waves in the α frequency band with eyes closed according to the first embodiment, in which the horizontal axis indicates a frequency and the vertical axis indicates a density. FIG. 23 is a graph illustrating a probability density distribution of an instantaneous phase difference of brain waves and pulse waves in the α frequency band with eyes closed according to the first embodiment, in which the horizontal axis indicates a phase difference and the vertical axis indicates a density.

As shown in FIGS. 12 to 23, instantaneous values measured at predetermined time intervals form a probability density distribution. Note that the probability density distribution is formed not only by instantaneous values of brain waves and pulse waves but also by instantaneous values of other pulse-related waves and brain-related waves. In this example, however, an instantaneous frequency of the brain waves, an instantaneous frequency of the pulse waves, and a probability density distribution of the instantaneous phase difference between the brain waves and the pulse waves are shown.

### <Fitting>

By fitting a probability density distribution of each instantaneous value by a predetermined function, feature amounts of the probability density distribution of instantaneous values can be calculated. For example, by fitting the probability density distribution of the instantaneous frequency by a Gaussian distribution, feature amounts such as a mean value, a variance value and the like can be obtained.

Further, by fitting the probability density distribution of the instantaneous phase difference by a Von Mises distribution, feature amounts such as a mean value, a degree of concentration and the like can be obtained. Since the value of the instantaneous phase difference is limited to be in a range of −π to +π, a Von Mises distribution is applied. The Von Mises distribution is expressed by the following Equation (15). Here, Iⱼ(κ) is the modified Bessel function of the first kind with order j of Equation (16).

$$f ( θ ) = exp ( κ cos ( θ - μ ) ) / 2 π {I}_{0} ( κ ) \qquad ( 15 )$$

$${I}_{j} ( κ ) = {( κ / 2 )}^{j} \sum {( {κ}^{2} / 4 )}^{i} / i ! Γ ( j + i + 1 ) \qquad ( 16 )$$

As shown in FIG. 12, when a probability density distribution of an instantaneous frequency of brain waves in the α frequency band with eyes open is fit by a Gaussian distribution, the mean value μ is 10.41 Hz and the variance value is 1.28. The measurement period is 70-90 seconds. As shown in FIG. 13, when a probability density distribution of an instantaneous frequency of pulse waves in the α frequency band with eyes open is fit by a Gaussian distribution, the mean value u is 9.71 Hz and the variance value is 1.07. The measurement period is 70-90 seconds. As shown in FIG. 14, when a probability density distribution of an instantaneous phase difference of brain waves and pulse waves in the α frequency band with eyes open is fit by a Von Mises distribution, the mean value is 0.13 Hz and the degree of concentration is 0. The measurement period is 70-90 seconds.

As shown in FIG. 15, when a probability density distribution of an instantaneous frequency of brain waves in the α frequency band with eyes open is fit by a Gaussian distribution, the mean value is 10.26 Hz and the variance value is 1.19. The measurement period is 210-230 seconds. As shown in FIG. 16, when a probability density distribution of an instantaneous frequency of pulse waves in the α frequency band with eyes open is fit by a Gaussian distribution, the mean value is 9.5 Hz and the variance value is 0.92. The measurement period is 210-230 seconds. As shown in FIG. 17, when a probability density distribution of an instantaneous phase difference of brain waves and pulse waves in the α frequency band with eyes open is fit by a Von Mises distribution, the mean value is 0.78 Hz and the degree of concentration is 0.13. The measurement period is 210-230 seconds.

As shown in FIG. 18, when a probability density distribution of an instantaneous frequency of brain waves in the α frequency band with eyes closed is fit by a Gaussian distribution, the mean value is 9.1 Hz and the variance value is 0.96. The measurement period is 340-360 seconds. As shown in FIG. 19, when a probability density distribution of an instantaneous frequency of pulse waves in the α frequency band with eyes closed is fit by a Gaussian distribution, the mean value is 8.99 Hz and the variance value is 1.07. The measurement period is 340-360 seconds. As shown in FIG. 20, when a probability density distribution of the instantaneous phase difference of brain waves and pulse waves in the α frequency band with eyes closed is fit by a Von Mises distribution, the mean value is 0.28 Hz and the degree of concentration is 0.29. The measurement period is 340-360 seconds.

As shown in FIG. 21, when a probability density distribution of an instantaneous frequency of brain waves in the α frequency band with eyes closed is fit by a Gaussian distribution, the mean value is 9.39 Hz and the variance value is 0.97. The measurement period is 410-430 seconds. As shown in FIG. 22, when a probability density distribution of the instantaneous frequency of pulse waves in the α frequency band with eyes closed is fit by a Gaussian distribution, the mean value is 9.03 Hz and the variance value is 0.98. The measurement period is 410-430 seconds. As shown in FIG. 23, when a probability density distribution of an instantaneous phase difference of brain waves and pulse waves in the α frequency band with eyes closed is fit by a Von Mises distribution, the mean value is 0.2 and the degree of concentration is 0.33. The measurement period is 410-430 seconds.

### <Tuning>

Next, tuning processing will be described. In the tuning processing, feature amounts that satisfy predetermined conditions are extracted from a relation between feature amounts of two different biological waves. In the following, the explanation will be given using examples of brain waves and pulse waves. The tuning processing extracts, for example, a case where the distribution of the instantaneous frequency of the brain waves and the distribution of the instantaneous frequency of the pulse waves are close to each other. The case where the distribution of the instantaneous frequency of the brain waves and the distribution of the instantaneous frequency of the pulse waves are close to each other is a case where the following Equation (17) is satisfied.

$$( \vert {μ}_{e} - {μ}_{b} \vert < ( {σ}_{e} + {σ}_{b} ) / η ) \qquad ( 17 )$$

Here, η is expressed, for example, as the following Equation (18).

$$η = 3 \qquad ( 18 )$$

In FIGS. 12 and 13, the left side of Equation (17) is established when |10.41−9.71|=0.7 and the right side of Equation (17) is established when (1.28+1.07)/3=0.783. In FIGS. 15 and 16, the left side of Equation (17) is not established when |10.26−9.5|=0.76 and the right side of Equation (17) is not established when (1.19+0.92)/3=0.703.

In FIGS. 18 and 19, the left side of Equation (17) is established when |9.1−8.99|=0.11 and the right side of Equation (17) is established when (0.96+1.07)/3=0.676. In FIGS. 21 and 22, the left side of Equation (15) is established when |9.39−9.03|=0.36 and the right side of Equation (17) is established when (0.97+0.98)/3=0.65. In this way, extracting instantaneous values of the brain waves and the pulse waves where Equation (17) is established are called tuning processing.

In the tuning processing, feature amounts when brain waves and pulse waves are correlated with each other are extracted. For example, pulse waves measured from vessels flowing into the brain such as carotid artery bifurcations or vertebral arteries are closely related to activities of brain waves. Therefore, by extracting feature amounts when Equation (17) is established, brain waves and pulse waves that are related to each other can be extracted.

### <Estimation of Physiological State>

Next, estimation of a physiological state will be described. In this example, a method for estimating the physiological state using brain waves and pulse waves will be described. The physiological state is estimated from feature amounts of instantaneous values of brain waves and pulse waves extracted by tuning processing. In the following, some examples of estimating the physiological state will be illustrated.

The feature amounts shown in FIGS. 12-14 and FIGS. 18-20 are the ones that satisfy Equation (17) and have been extracted by tuning processing. As shown in FIGS. 12 and 13, in a probability density distribution of an instantaneous frequency in the α frequency band with eyes open, the mean value of the brain waves is 10.41 and the mean value of the pulse waves is 9.71. Therefore, they are both about 10 Hz. On the other hand, as shown in FIGS. 18 and 19, in a probability density distribution of an instantaneous frequency in the α frequency band with eyes closed, the mean value of the brain waves is 9.1 and the mean value of the pulse waves is 8.99. Therefore, they are both about 9 Hz. In this way, as the state in which eyes are open has been changed to the state in which eyes are closed, the mean value of the brain waves and that of the pulse waves are shifted from about 10 Hz to about 9 Hz.

Further, as shown in FIG. 14, in a probability density distribution of an instantaneous phase difference of brain waves and pulse waves in the α frequency band with eyes open, the degree of concentration is 0. On the other hand, as shown in FIG. 20, in a probability density distribution of an instantaneous phase difference of brain waves and pulse waves in the α frequency band with eyes closed, the degree of concentration is 0. 29. In this way, as the state in which eyes are open has been changed to the state in which eyes are closed, a degree of concentration of an instantaneous phase difference between brain waves and pulse waves increases from 0 to 0.29.

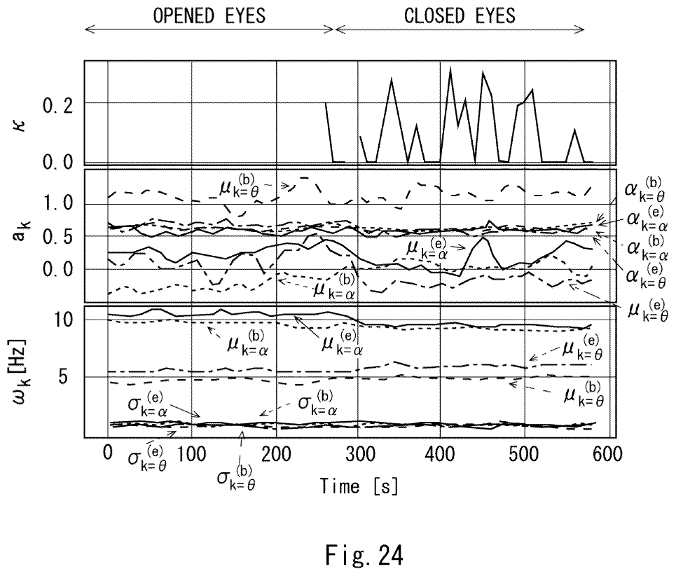

FIG. 24 is a graph illustrating feature amounts when the eyes are open and when the eyes are closed according to the first embodiment, in which the horizontal axis indicates a time and the vertical axis indicates a feature amount of an instantaneous frequency, a feature amount of an instantaneous logarithmic amplitude, and a degree of concentration. As described above, as the state in which eyes are open has been changed to the state in which eyes are closed, the mean value of the instantaneous frequency of the brain waves and the mean value of the instantaneous frequency of the pulse waves are shifted from about 10 Hz to about 9 Hz. Accordingly, the probability density distribution of the instantaneous frequency of the brain waves and that of the pulse waves overlap each other. Therefore, as shown in FIG. 24, the degree of concentration κ becomes larger than 0. This indicates that, when the eyes are open, pulse waves measured from the carotid artery bifurcation are not synchronized with brain waves because multiple centers are active, whereas when the eyes are closed, pulse waves measured from the carotid artery bifurcation are synchronized with brain waves because fewer centers are active. In this manner, it is possible to estimate the state of the centers that are active in the brain due to changes in feature amounts.

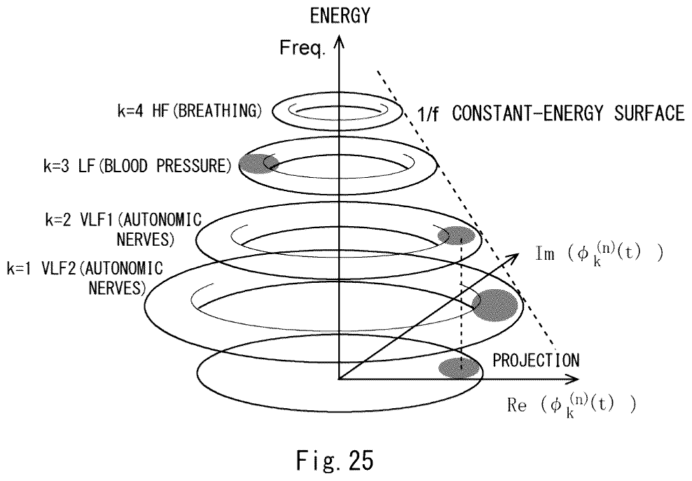

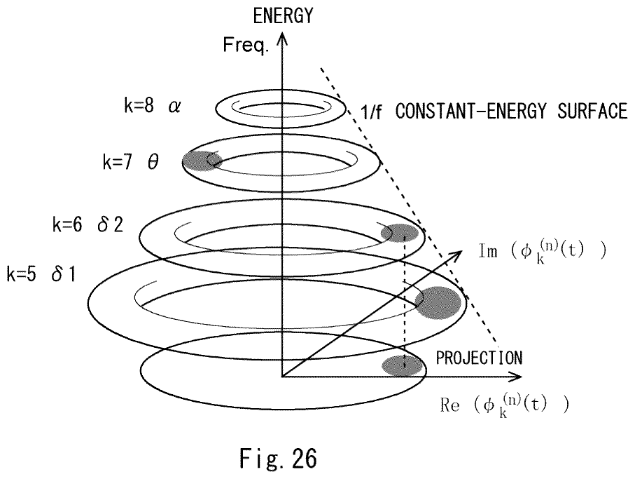

FIGS. 25 and 26 are diagrams each illustrating a complex waveform equation in each frequency band in a three dimensional manner according to the first embodiment, in which the horizontal axis and the vertical axis on a complex plane indicate Re(lnφₖ⁽ⁿ⁾(t)) and Im(lnφₖ⁽ⁿ⁾(t)) of a complex transformation expression and an axis orthogonal to the complex plane indicates α frequency band (energy axis). For example, k=4 in α frequency band longer than the pulse interval is expressed as HF (breathing), k=3 is expressed as LF (blood pressure), k=2 is expressed as VLF1 (it may be related to autonomic nerves), and k=1 is expressed as VLF2 (it may be related to autonomic nerves). k=5 in a brain wave band is expressed as δ1, k=6 is expressed as δ2, k=7 is expressed as θ, and k=8 is expressed as α. Then, the moving radius in the complex plane is an instantaneous logarithmic amplitude of biological waves in each frequency band. The rotational direction about the energy axis is an instantaneous phase of biological waves in each frequency band. The energy axis is α frequency band axis. Then, frequency bands are rotated about the energy axis while they are distributed like a Gaussian distribution in each axis. In this way, a drawing can be obtained in which electrons of each energy level orbit around the nucleus as if they were an electron cloud expressed in terms of the probability of existence. The Gaussian-like distribution of each frequency band, similar to electron clouds, is formed along the constant-energy surface of 1/F.

Accordingly, when there are instantaneous values (feature amounts) in frequency bands which are deviated from a Gaussian distribution which is along the constant-energy surface of 1/F, it can be estimated that there is an abnormality in a physiological state characteristic of this frequency band. For example, when instantaneous values (feature amounts) of VLF1 and VLF2 are deviated from a Gaussian distribution that is along the constant-energy surface of 1/F, it can be estimated that there is an abnormality in a physiological state related to the autonomic nerves.

### <Physiological State Estimation System>

Next, a physiological state estimation system according to this embodiment will be described. The physiological state estimation system according to this embodiment includes a physiological state estimation apparatus. In the following, a physiological state estimation apparatus will be described as the physiological state estimation system.

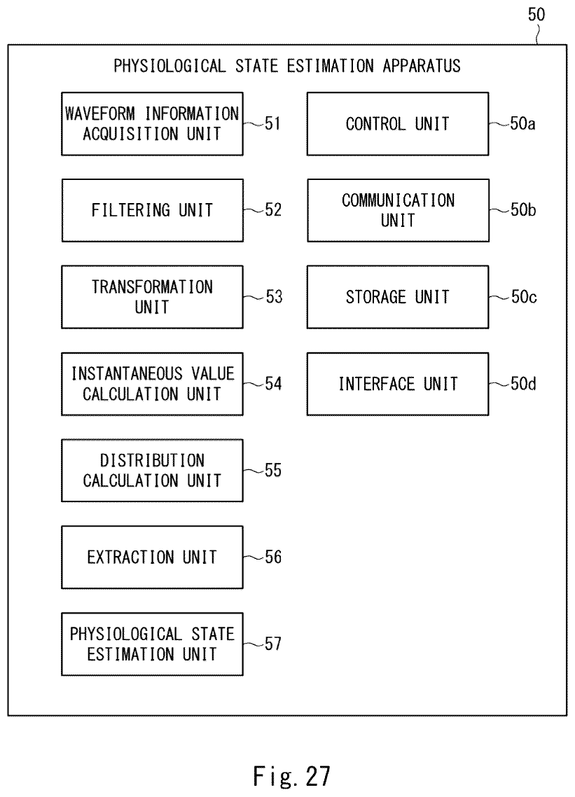

FIG. 27 is a block diagram illustrating the physiological state estimation apparatus according to the first embodiment. As shown in FIG. 27, the physiological state estimation apparatus 50 includes a control unit 50a, a communication unit 50b, a storage unit 50c, an interface unit 50d, a waveform information acquisition unit 51, a filtering unit 52, a transformation unit 53, an instantaneous value calculation unit 54, a distribution calculation unit 55, an extraction unit 56, and a physiological state estimation unit 57. The control unit 50a, the communication unit 50b, the storage unit 50c, the interface unit 50d, the waveform information acquisition unit 51, the filtering unit 52, the transformation unit 53, the instantaneous value calculation unit 54, the distribution calculation unit 55, the extraction unit 56, and the physiological state estimation unit 57 respectively function as control means, communication means, storage means, interface means, waveform information acquisition means, filtering means, transformation means, instantaneous value calculation means, distribution calculation means, extraction means, and physiological state estimation means.

The physiological state estimation apparatus 50 is an information processing apparatus including a computer. The control unit 50a includes, for example, a processor such as a Central Processing Unit (CPU), a Micro Processing Unit (MPU), an Electronic Control Unit (ECU), a Field-Programmable Gate Array (FPGA), or an Application Specific Integrated Circuit (ASIC). The control unit 50a includes a function as an arithmetic apparatus that performs control processing, arithmetic processing, and so on. Further, the control unit 50a controls the operations of the respective components of the communication unit 50b, the storage unit 50c, the interface unit 50d, the waveform information acquisition unit 51, the filtering unit 52, the transformation unit 53, the instantaneous value calculation unit 54, the distribution calculation unit 55, the extraction unit 56, and the physiological state estimation unit 57, and so on.

Each of the components of the physiological state estimation apparatus 50 can be implemented, for example, by executing a program through control performed by the control unit 50a. More specifically, each of the components may be implemented by the control unit 50a executing the program stored in the storage unit 50c. Further, by recording a necessary program in any non-volatile storage medium and installing the program as necessary, each of the components may be implemented. Further, each of the components is not limited to be implemented by software by a program and may be implemented by, for example, any combination of hardware, firmware, and software.

The communication unit 50b receives waveform information of biological waves measured by a waveform measurement device such as the brain wave measurement device 10 and the pulse wave measurement device 20 from the waveform measurement device. The waveform information of the biological waves includes, for example, time-series data of biological waves.

The storage unit 50c may include, for example, a storage apparatus such as a memory or a hard disk. The storage apparatus is, for example, a Read Only Memory (ROM) or a Random Access Memory (RAM). The storage unit 50c includes a function for storing a control program, an arithmetic program and the like executed by the control unit 50a. Further, the storage unit 50c includes a function for temporarily storing process data and so on. Further, the storage unit 50c stores a correspondence between feature amounts in instantaneous values of biological waves of a subject performed in advance and a physiological state of the subject. The storage unit 50c may store waveform information of the biological waves received by the communication unit 50b.

The interface unit 50d is, for example, a user interface. The interface unit 50d includes an input apparatus such as a keyboard, a touch panel, or a mouse, and an output apparatus such as a display or a speaker. The interface unit 50d accepts an operation of inputting data performed by a user (an operator or the like) and outputs information to the user.

The waveform information acquisition unit 51 acquires the waveform information that the communication unit 50b has acquired from the waveform measurement device. The waveform information is, for example, time-series data of biological waves. The waveform information acquisition unit 51 acquires, for example, brain waves and pulse waves. The waveform information acquisition unit 51 may include a pulse-related wave acquisition unit configured to acquire pulse-related waves and a brain-related wave acquisition unit configured to acquire brain-related waves.

The filtering unit 52 filters the acquired waveform information in at least one predetermined frequency band.

The transformation unit 53 performs transformation for transforming the waveform information filtered in the frequency band into a complex number. The transformation for transforming the waveform information into a complex number is, for example, Hilbert transformation.

The instantaneous value calculation unit 54 calculates instantaneous values. As described above, the instantaneous values include the instantaneous logarithmic amplitude aₖ⁽ⁿ⁾(t), the instantaneous phase ψₖ⁽ⁿ⁾(t), the instantaneous frequency ωₖ⁽ⁿ⁾(t), and the instantaneous phase difference θₖ^{(x, y)}(t). The instantaneous value calculation unit 54 calculates, for example, instantaneous values including an instantaneous logarithmic amplitude aₖ⁽ⁿ⁾(t) corresponding to a logarithm of an amplitude term of a complex waveform equation obtained by performing Hilbert transformation, and an instantaneous frequency ωₖ⁽ⁿ⁾(t) corresponding to a time differential value of a phase term of the complex waveform equation.

The distribution calculation unit 55 calculates a probability density distribution of instantaneous values. Then, the distribution calculation unit 55 calculates feature amounts including a mean value and a variance value of the probability density distribution of the instantaneous values. Note that the distribution calculation unit 55 may cause a Gaussian distribution to be fit to the probability density distribution depending on the instantaneous values. In this case, a mean value and a variance value of the probability density distribution are a mean value and a variance value when the probability density distribution is fit by a Gaussian distribution. Further, the distribution calculation unit 55 may cause a Von Mises distribution to be fit to the probability density distribution depending on the instantaneous values. In this case, the mean value of the probability density distribution is a mean value when the probability density distribution is fit by a Von Mises distribution.

The extraction unit 56 extracts instantaneous values when predetermined conditions are met. The predetermined conditions are, for example, the following conditions. That is, when a mean value of an instantaneous frequency of the brain waves in a predetermined frequency band is denoted by μₑ, a variance value of an instantaneous frequency of the brain waves in a predetermined frequency band is denoted by σₑ, a mean value of an instantaneous frequency of the pulse waves in a predetermined frequency band is denoted by μb, a variance value of an instantaneous frequency of the pulse waves in a predetermined frequency band is denoted by σ_{b}, and a parameter of an integer equal to or greater than 3 is denoted by f, the extraction unit 56 extracts instantaneous values when the above-described Equation (17) is satisfied. η of the parameter is, for example, 3.

The physiological state estimation unit 58 estimates a physiological state from feature amounts of the instantaneous values that have been extracted. For example, feature amounts calculated for biological waves of the subject and the physiological state of the subject such as subjective evaluation RAS and stress scale PSS are associated with each other in advance. Then, the physiological state of the subject is estimated from feature amounts of the instantaneous values that have been extracted.

### <Physiological State Estimation Method>

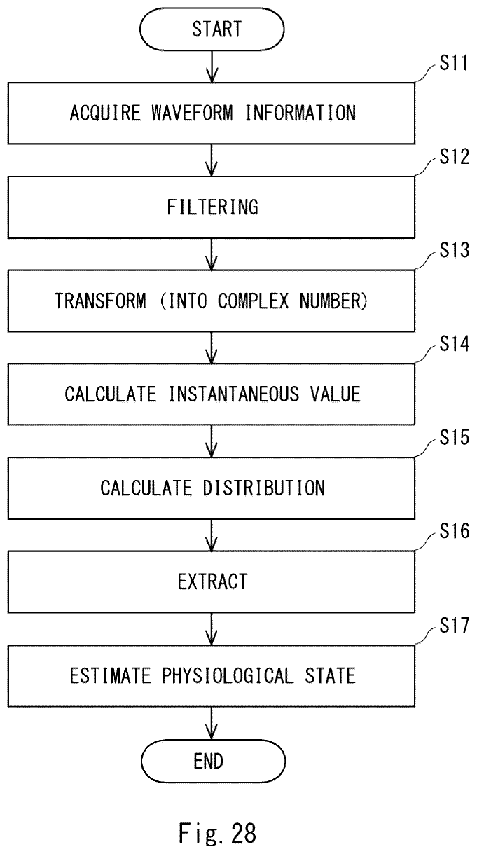

Next, a physiological state estimation method will be described. FIG. 28 is a flowchart illustrating a physiological state estimation method according to the first embodiment. As shown in FIG. 28, the physiological state estimation method includes a waveform information acquisition step of acquiring waveform information on a living body (Step S11), a filtering step of filtering the acquired waveform information in at least one frequency band (Step S12), a transformation step of transforming the waveform information filtered in the frequency band into a complex waveform equation (Step S13), an instantaneous value calculation step of calculating an instantaneous value including at least one of an instantaneous logarithmic amplitude, an instantaneous frequency, an instantaneous phase, and an instantaneous phase difference (Step S14), a distribution calculation step of calculating a distribution of instantaneous values (Step S15), an extraction step of extracting instantaneous values when predetermined conditions are met (Step S16), and a physiological state estimation step of estimating a physiological state (Step S17).

### <Waveform Information Acquisition Step>

First, in the waveform information acquisition step (Step S11), the waveform information acquisition unit 51 acquires waveform information of biological waves such as brain waves and pulse waves. More specifically, the waveform information acquisition unit 51 acquires brain waves measured by the brain wave measurement device 10 and pulse waves measured by the pulse wave measurement device 20.

### <Filtering Step>

Next, in the filtering step (Step S12), the filtering unit 52 filters the acquired waveform information in at least one predetermined frequency band.

### <Transformation Step>

Next, in the transformation step (Step S13), the transformation unit 53 transforms the time-series data φₖ⁽ⁿ⁾(t) filtered in the frequency band into a complex waveform equation by Hilbert transformation.

### <Instantaneous Value Calculation Step>

Next, in the instantaneous value calculation step (Step S14), the instantaneous value calculation unit 54 calculates the aforementioned instantaneous values. Specifically, the instantaneous value calculation unit 54 calculates an instantaneous value including at least one of an instantaneous logarithmic amplitude corresponding to a logarithm of an amplitude term of a complex waveform equation obtained by performing Hilbert transformation, an instantaneous phase corresponding to a phase term of the complex waveform equation, an instantaneous frequency corresponding to a time differential value of a phase term of a complex waveform equation, and an instantaneous phase difference corresponding to a difference between respective instantaneous phases of two different biological waves.

### <Instantaneous Value Distribution Calculation Step>

Next, in the instantaneous value distribution calculation step (Step S15), the distribution calculation unit 55 calculates a distribution of the instantaneous values. Specifically, the distribution calculation unit 55 calculates a probability density distribution of an instantaneous logarithmic amplitude, an instantaneous phase, an instantaneous frequency, and an instantaneous phase difference in a desired time interval. Then, the distribution calculation unit 55 calculates, for example, a mean value and a variance value as the aforementioned feature amounts of the probability density distribution.

### <Extraction Step>

Next, in the extraction step (Step S16), the extraction unit 56 extracts instantaneous values when Equation (17) is met when a mean value of an instantaneous frequency of the brain waves in a predetermined frequency band is denoted by μₑ, a variance value of an instantaneous frequency of the brain waves in a predetermined frequency band is denoted by σₑ, a mean value of an instantaneous frequency of the pulse waves in a predetermined frequency band is denoted by μ_{b}, a variance value of an instantaneous frequency of the pulse waves in a predetermined frequency band is denoted by σ_{b}, and a parameter of an integer equal to or greater than 3 is denoted by n.

### <Physiological State Estimation Step>

Next, in the physiological state estimation step (Step S17), the physiological state estimation unit 57 estimates the physiological state from feature amounts of the instantaneous values that have been extracted.

Next, effects of this embodiment will be described. In this embodiment, instantaneous values which satisfy predetermined conditions in which brain waves and pulse waves are correlated with each other are extracted to estimate a physiological state from feature amounts of the instantaneous values that have been extracted. Therefore, it is possible to estimate the physiological state with a high accuracy. Further, since the brain waves and the pulse waves are filtered in a specific frequency band correlated with the physiological state, it is possible to estimate the physiological state related to the above specific frequency band with a high accuracy. Since the probability density distributions of the instantaneous logarithmic amplitude, the instantaneous phase, and the instantaneous frequency are fit by a Gaussian distribution, a mean value and a variance value can be acquired as feature amounts. Further, since the probability density distribution of the instantaneous phase difference is fit by a Von Mises distribution, a mean value and a degree of concentration can be acquired as feature amounts. It is therefore possible to estimate the physiological state with a high accuracy.

Note that the present disclosure is not limited to the above-described embodiments and may be changed as appropriate without departing from the spirit of the present disclosure. For example, a physiological state estimation program causing a computer to execute a physiological state estimation method is also included in the scope of the technical idea of the embodiments.

A (The) program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A physiological state estimation system comprising:
a waveform information acquisition unit configured to acquire brain waves and pulse waves;
a filtering unit configured to filter the acquired brain waves and pulse waves in at least one predetermined frequency band;
a transformation unit configured to perform Hilbert transformation on the brain waves and pulse waves filtered in the frequency band;
an instantaneous value calculation unit configured to calculate instantaneous values including an instantaneous logarithmic amplitude corresponding to a logarithm of an amplitude term of a complex waveform equation obtained by performing Hilbert transformation and an instantaneous frequency corresponding to a time differential value of a phase term of the complex waveform equation;
a distribution calculation unit configured to calculate a probability density distribution of the instantaneous values and calculate feature amounts including a mean value and a variance value of the probability density distribution;
an extraction unit configured to extract the instantaneous values when
$$\vert {μ}_{e} - {μ}_{b} \vert < ( {σ}_{e} + {σ}_{b} ) / η$$
is met, where a mean value of the instantaneous frequency of the brain waves in the predetermined frequency band is denoted by μₑ,
a variance value of the instantaneous frequency of the brain waves in the predetermined frequency band is denoted by σₑ,
a mean value of the instantaneous frequency of the pulse waves in the predetermined frequency band is denoted by μ_{b},
a variance value of the instantaneous frequency of the pulse waves in the predetermined frequency band is denoted by σ_{b}, and
a parameter of an integer equal to or greater than 3 is denoted by η; and
a physiological state estimation unit configured to estimate a physiological state from the feature amounts of the instantaneous values that have been extracted.

2. The physiological state estimation system according to claim 1, wherein f of the parameter is 3.

3. A physiological state estimation method comprising:
acquiring brain waves and pulse waves;
filtering the acquired brain waves and pulse waves in at least one predetermined frequency band;
performing Hilbert transformation on the brain waves and pulse waves filtered in the frequency band;
calculating instantaneous values including an instantaneous logarithmic amplitude corresponding to a logarithm of an amplitude term of a complex waveform equation obtained by performing Hilbert transformation and an instantaneous frequency corresponding to a time differential value of a phase term of the complex waveform equation;
calculating a probability density distribution of the instantaneous values and calculating feature amounts including a mean value and a variance value of the probability density distribution;
extracting the instantaneous values when
$$\vert {μ}_{e} - {μ}_{b} \vert < ( {σ}_{e} + {σ}_{b} ) / η$$
is met, where a mean value of the instantaneous frequency of the brain waves in the predetermined frequency band is denoted by μₑ,
a variance value of the instantaneous frequency of the brain waves in the predetermined frequency band is denoted by σₑ,
a mean value of the instantaneous frequency of the pulse waves in the predetermined frequency band is denoted by μ_{b},
a variance value of the instantaneous frequency of the pulse waves in the predetermined frequency band is denoted by σ_{b}, and
a parameter of an integer equal to or greater than 3 is denoted by η; and
estimating a physiological state from the feature amounts of the instantaneous values that have been extracted.

4. A non-transitory computer readable medium storing a physiological state estimation program causing a computer to execute:
acquiring brain waves and pulse waves;
filtering the acquired brain waves and pulse waves in at least one predetermined frequency band;
performing Hilbert transformation on the brain waves and pulse waves filtered in the frequency band;
calculating instantaneous values including an instantaneous logarithmic amplitude corresponding to a logarithm of an amplitude term of a complex waveform equation obtained by performing Hilbert transformation and an instantaneous frequency corresponding to a time differential value of a phase term of the complex waveform equation;
calculating a probability density distribution of the instantaneous values and calculating feature amounts including a mean value and a variance value of the probability density distribution;
extracting the instantaneous values when
$$\vert {μ}_{e} - {μ}_{b} \vert < ( {σ}_{e} + {σ}_{b} ) / η$$
is met, where a mean value of the instantaneous frequency of the brain waves in the predetermined frequency band is denoted by μₑ,
a variance value of the instantaneous frequency of the brain waves in the predetermined frequency band is denoted by σₑ,
a mean value of the instantaneous frequency of the pulse waves in the predetermined frequency band is denoted by μ_{b},
a variance value of the instantaneous frequency of the pulse waves in the predetermined frequency band is denoted by σ_{b}, and
a parameter of an integer equal to or greater than 3 is denoted by η; and
estimating a physiological state from the feature amounts of the instantaneous values that have been extracted.
